(19) United States
(12) Patent Application Publication
(10) Pub. No.: US 2013/0011886 A1
(43) Pub. Date: Jan. 10, 2013

(54) METHOD FOR THE PRODUCTION OF A FERMENTATION PRODUCT FROM LIGNOCELLULOSIC FEEDSTOCKS

(75) Inventors: Jeffrey S. Tolan, Ottawa (CA); Brian Foody, Ottawa (CA)

(73) Assignee: IOGEN ENERGY CORPORATION, Ottawa, ON (CA)

(21) Appl. No.: 13/522,099

(22) PCT Filed: Feb. 10, 2011
(86) PCT No.: PCT/CA11/00163

§ 371 Date: Sep. 25, 2012

Related U.S. Application Data
(60) Provisional application No. 61/303,030, filed on Feb. 10, 2010.

(51) Int. Cl. C12P 7/14; C12P 7/56; C12P 7/54; C12P 7/48; C12P 7/02; C12P 7/40; C12P 7/42; C12P 7/44; C12P 17/04; C12P 7/16; C12P 7/46

(52) U.S. Cl. 435/126; 435/162; 435/160; 435/139; 435/140; 435/144; 435/145; 435/136; 435/146; 435/142; 435/155

(57) ABSTRACT

The present invention comprises pretreating a lignocellulosic feedstock with acid at a pH between about 2.0 and about 3.5 to produce a composition comprising an acid pretreated feedstock. The acid pretreated feedstock is then enzymatically hydrolyzed with cellulases and β-glucosidase. The glucose is fermented by microorganisms to produce a fermentation broth comprising the fermentation product, followed by recovery of the fermentation product. The steps of enzymatically hydrolyzing and fermenting are conducted at a pH below about 4.0.

The present invention relates to a method for producing a fermentation product from a lignocellulosic feedstock. More specifically, the present invention relates to a method for producing a fermentation product from a lignocellulosic feedstock involving acid pretreatment and cellulose hydrolysis.

### BACKGROUND OF THE INVENTION

Plant cell walls consist mainly of the large biopolymers cellulose, hemicellulose, lignin and pectin. Cellulose consists of D-glucose units linked together in linear chains via beta-1,4 glycosidic bonds. Hemicellulose consists primarily of a linear xylan backbone comprising D-xylose units linked together via beta-1,4 glycosidic bonds and numerous side chains linked to the xylose units via beta-1,2 or beta-1,3 glycosidic or ester bonds (e.g. L-arabinose, acetic acid, ferulic acid, etc.).

Lignocellulosic feedstock is a term commonly used to describe plant-derived biomass comprising cellulose, hemicellulose and lignin. Much attention and effort has been applied in recent years to the production of fuels and chemicals, primarily ethanol, from lignocellulosic feedstocks, such as agricultural wastes and forestry wastes, due to their low cost and wide availability. These agricultural and forestry wastes are typically burned and landfilled; thus, using these lignocellulosic feedstocks for ethanol production offers an attractive alternative to disposal. Yet another advantage of these feedstocks is that the lignin byproduct, which remains after the cellulose conversion process, can be used as a fuel to power the process instead of fossil fuels. Several studies have concluded that, when the entire production and consumption cycle is taken into account, the use of ethanol produced from cellulose generates close to zero greenhouse gases.

In comparison, fuel ethanol from feedstocks such as corn starch, sugar cane and sugar beets suffers from the limitation that these feedstocks are already in use as a food source for animals and humans. A further disadvantage of the use of these feedstocks is that fossil fuels are used in the conversion processes. Thus, these processes have only a limited impact on reducing greenhouse gases.

Lignocellulosic feedstocks have also been considered for producing other products besides ethanol. For example, lactic acid has received much attention in recent years for the production of biodegradable lactide polymers. It is expected that this biodegradable polymer, produced from renewable resources, will partially replace various petrochemical-based polymers in applications ranging from packaging to clothing (van Maris et al., 2004, Microbial Export of Lactic and 3-Hydroxypropanoic Acid: Implications for Industrial Fermentation Processes, In Metabolic engineering of pyruvate metabolism in Saccharomyces cerevisiae, Ed. Van Maris, ppg 79-97).

The first chemical processing step for converting lignocellulosic feedstock to ethanol or other fermentation products involves hydrolysis of the cellulose and hemicellulose polymers to sugar monomers, such as glucose and xylose, which can be converted to ethanol or other fermentation products in a subsequent fermentation step. Hydrolysis of the cellulose and hemicellulose can be achieved with a single-step chemical treatment or with a two-step process with milder chemical pretreatment followed by enzymatic hydrolysis of the pretreated lignocellulosic feedstock with cellulase enzymes.

In a single-step chemical treatment, the lignocellulosic feedstock is contacted with a strong acid or alkali under conditions sufficient to hydrolyze both the cellulose and hemicellulose components of the feedstock to sugar monomers.

In the two-step chemi-enzymatic hydrolysis process, the lignocellulosic feedstock is first subjected to a pretreatment under conditions that are similar to, but milder than, those in the single-step acid or alkali hydrolysis process. The purpose of the pretreatment is to increase the cellulose surface area and convert the fibrous feedstock to a muddy texture, with limited conversion of the cellulose to glucose. If the pretreatment is conducted with acid, the hemicellulose component of the feedstock is hydrolyzed to xylose, arabinose, galactose and mannose. The resulting hydrolyzate, which is enriched in pentose sugars derived from the hemicellulose, may be separated from the solids and used in a subsequent fermentation process to convert the pentose sugars to ethanol or other products.

After the pretreatment step, the cellulose is subjected to enzymatic hydrolysis with one or more cellulase enzymes such as exo-cellobiohydrolases (CBH), endoglucanases (EG) and beta-glucosidases. The CBH and EG enzymes catalyze the hydrolysis of the cellulose (β-1,4-D-glucan linkages). The CBH enzymes, CBHI and CBHII, act on the ends of the glucose polymers in cellulose microfibrils and liberate cellobiose, while the EG enzymes act at random locations on the cellulose. Together, the cellulase enzymes hydrolyze cellulose to cellobiose, which, in turn, is hydrolyzed to glucose by beta-glucosidase (beta-G).

If glucose is the predominant sugar present in the hydrolyzate, the fermentation is typically carried out with a Saccharomyces spp. strain. However, if the hydrolyzate comprises significant proportions of xylose and arabinose carried through from the pretreatment, the fermentation is conducted with a microbe that naturally contains, or has been engineered to contain, the ability to ferment xylose and/or arabinose to ethanol or other product(s). Examples of microbes that have been genetically modified to ferment xylose include recombinant Saccharomyces strains into which has been inserted either (a) the xylose reductase (XR) and xylitol dehydrogenase (XDH) genes from Pichia stipitis (U.S. Pat. Nos. 5,789,210, 5,866,382, 6,582,944 and 7,527,927 and EP 450 530) or (b) fungal or bacterial xylose isomerase (XI) gene (U.S. Pat. Nos. 6,475,768 and 7,622,284).

Ethanol recovery from the fermented solution is typically carried out by distillation, which involves pumping the broth through one or more distillation columns to separate the ethanol from the other components in the broth. In a conventional distillation process, dilute beer is sent to a beer column where it is partially concentrated and ethanol-enriched vapour from the beer column is sent to a rectification column for further purification. After distillation, the small amounts of water remaining may be removed from the vapour by a molecular sieve resin, by membrane extraction or other expedients.

Each stage of the lignocellulosic conversion process is carried out at a pH range at which the chemical or biological reaction operates most efficiently. The pH typical of the lignocellulosic feedstock fed to the process and the pH ranges for each processing step to produce ethanol, namely acid pretreatment, enzymatic hydrolysis, fermentation and distillation, are shown in FIG. 1.

As shown in FIG. 1, the pH of the incoming feedstock is between about 6.0 and 10.0 and then is decreased with acid to a pH between about 0.5 and 2.0, which is a conventional pH range for acid pretreatment (see WO 2006/128304). After pretreatment, alkali is added to the acidic, pretreated feedstock to achieve the optimal pH range of 4.5 to 5.5 for cellulase enzymes. The pH of the glucose stream resulting from enzymatic hydrolysis may be subsequently adjusted to a value that is amenable to most fermentations and this is usually between 4 and 5.5 for the yeast that are commonly used in this stage, such as Saccharomyces cerevisiae. The feed to the step of product recovery, which is distillation in the case of ethanol, is generally at a pH between 4.0 and 5.5 and thus the ethanol-containing feed stream (known as “beer”) may or may not require a pH adjustment.

One drawback of conventional processes is that significant amounts of acid and alkali are required during the conversion process to attain the pH ranges that are considered optimal for each stage. The high chemical demand for carrying out the pH adjustments at various stages of the process can significantly increase the cost. Compounding this, the addition of acid or alkali during the pH adjustments produces inorganic salts as a consequence of the neutralization of alkali or acid added in previous stages. This further increases the cost of the process as these salts must be processed and disposed of.

Acid pretreatment is one stage of the process that has a particularly high acid demand. The feedstock has a pH of between 6 and 10 due to the presence of the alkali minerals such as potassium carbonate, sodium carbonate, calcium carbonate and magnesium carbonate, and thus requires the addition of significant amounts of acid to adjust the pH of the feedstock down to values between 0.5 and 2.0. The minerals have a neutralizing effect on the pretreatment acid (Esteghlalian et al., 1997, Bioresource Technology, 59:129-136). For instance, sulfuric acid reacts with the cations of the carbonate salts during pretreatment to form calcium sulfate, magnesium sulfate, potassium sulfate and sodium sulfate. Bisulfate salts form as the pH is lowered further. Due to the presence of these minerals, additional acid is required to overcome the resistance of the feedstock to changes in pH, which further contributes to the chemical requirements of this stage.

A further drawback of acid pretreatment is that the low pH values utilized at this stage require the use of expensive acid-resistant materials on the pretreatment reactor and other downstream process equipment exposed to the acid pretreated feedstock. As well, sugars present in the pretreated feedstock (mainly xylose, glucose and arabinose) tend to degrade under such harshly acidic pH values.

The pH adjustment conducted to increase the pH of the acidic, pretreated feedstock to between 4.5 and 5.5 prior to enzymatic hydrolysis with cellulase enzymes also contributes to the high chemical demand of the process due to the presence of acetic acid that arises from the hydrolysis of acetyl groups from hemicellulose during acid pretreatment. Notably, the pKa of acetic acid is 4.75 and, at a pH corresponding to its pKa, the buffering capacity of this weak acid is at its maximum. Thus, when the acidic, pretreated feedstock is increased from a pH between 0.5 and 2.0 to a pH between 4.5 and 5.5 for enzymatic hydrolysis, significant amounts alkali must be added to overcome the buffering effect of this weak organic acid. High levels of alkali addition also produce large amounts of salts as the alkali reacts with the acid in the pretreated feedstock.

The pH adjustment prior to fermentation to produce ethanol may also necessitate the addition of acid or alkali to adjust the pH of the glucose stream to the optimal pH of the microbes. As acetate and acetic acid arising from acid pretreatment will also be present in the glucose stream, the buffering effect will again need to be overcome to adjust the pH.

U.S. Pat. No. 5,424,417 (Torget et al.) discloses an acid prehydrolysis of a lignocellulosic feedstock utilizing mild conditions. This includes conducting the prehydrolysis at a pH in the range of 3-4 and at a temperature of 160° C. in a flow-through reactor in which fluid passes through the lignocellulosic material as hydrolysis proceeds so that hydrolyzed compounds are carried away with the flow of liquid. During the prehydrolysis, xylose oligomers may be removed and further treated in an additional hydrolysis stage to yield xylose monomers.

U.S. Pat. No. 4,168,988. (Riehm et al.) discloses solubilizing, dissolving and extracting salts from the residues of annuals by an aqueous acid solution. This is followed by hydrolyzing the pentosans in the acidified residues.

### SUMMARY OF THE INVENTION

The present invention overcomes several disadvantages of the prior art by taking into account the difficulties encountered in steps carried out during the processing of lignocellulosic feedstock to obtain a fermentation product.

It is an object of the invention to provide an improved method for producing a fermentation product from a lignocellulosic feedstock.

According to a first aspect of the invention, there is provided a method for obtaining a fermentation product from a lignocellulosic feedstock comprising: (i) pretreating the lignocellulosic feedstock with acid at a pH between about 2.0 and about 3.5 to produce a composition comprising an acid pretreated feedstock; (ii) enzymatically hydrolyzing the acid pretreated feedstock with cellulases and β-glucosidase to produce glucose; (iii) fermenting the glucose so produced with microorganisms to produce a fermentation broth comprising the fermentation product; and (iv) recovering the fermentation product from the fermentation broth, wherein the pH during each of the steps of enzymatically hydrolyzing, fermenting and recovering is between about pH 3.0 and about 4.0 and wherein the pH during fermenting is greater than or equal to the pH during enzymatically hydrolyzing and the pH during recovering is greater than or equal to the pH during the fermenting.

According to a second aspect of the invention, there is provided a method for obtaining a fermentation product from a lignocellulosic feedstock comprising: (i) pretreating the lignocellulosic feedstock with acid at a pH between about 2.5 and about 3.5 to produce a composition comprising an acid pretreated feedstock; (ii) enzymatically hydrolyzing the acid pretreated feedstock with cellulases and β-glucosidase to produce glucose; (iii) fermenting the glucose so produced with microorganisms to produce a fermentation broth comprising the fermentation product; and (iv) recovering the fermentation product from the fermentation broth, wherein the pH during each of the steps of enzymatically hydrolyzing, fermenting and recovering is between about pH 3.0 and about 4.0.

According to a third aspect of the invention, there is provided a method for obtaining a fermentation product from a lignocellulosic feedstock comprising: (i) pretreating the lignocellulosic feedstock with acid at a pH between about 2.0 and about 3.5 to produce a composition comprising an acid pretreated feedstock; (ii) enzymatically hydrolyzing the acid pretreated feedstock with cellulases and β-glucosidase to produce glucose; (iii) fermenting the glucose so produced with microorganisms to produce a fermentation broth comprising the fermentation product, wherein the pH during each of the steps of enzymatically hydrolyzing and fermenting is between about 3.0 and about 4.0.

According to a fourth aspect of the invention, there is provided a method for obtaining ethanol from a lignocellulosic feedstock comprising: (i) pretreating the lignocellulosic feedstock with acid at a pH between about 2.0 and about 2.5 to produce a composition comprising an acid pretreated feedstock; (ii) enzymatically hydrolyzing the acid pretreated with cellulases and β-glucosidase to produce glucose; and (iii) fermenting the glucose so produced with Saccharomyces cerevisiae to produce a fermentation broth comprising the ethanol, wherein the steps of enzymatically hydrolyzing and fermenting are each conducted at a pH range between about 3.5 and about 4.0 and wherein the pH during fermenting is greater than or equal to the pH during enzymatic hydrolysis.

The steps of pretreating, enzymatically hydrolyzing and fermenting are conducted in the order presented.

The present invention can provide numerous benefits over conventional processes for converting lignocellulosic feedstock to a fermentation product. By conducting the acid pretreatment at a higher pH than in prior processes, the economics of the process are improved. Minerals such as alkali carbonates native to the feedstock resist changes to the pH of the feedstock and thus conducting the acid pretreatment at pH values that are higher than what is considered conventional can lead to significant acid savings. Moreover, at higher pH values, the metallurgy of the pretreatment reactor and downstream process equipment exposed to acid pretreated feedstock may not need to be acid-resistant, which reduces expense. Additionally, less xylose degradation occurs at higher pH values, which in turn, can improve the xylose yield from acid pretreatment.

By conducting the enzymatic hydrolysis of the acid pretreated feedstock at a pH of less than 4.0, rather than the conventional pH of between 4.5 and 5.5, significantly less alkali is required to increase the pH of the pretreated feedstock. As set forth previously, during acid pretreatment, acetic acid is released from the hemicellulose component of the feedstock. In conventional processes in which the pH of enzymatic hydrolysis is between 4.5 and 5.5, in order to achieve this pH, large amounts of alkali are required to overcome the buffering effect of acetic acid in the acid pretreated feedstock, which reaches its maximum at a pH corresponding to its pKa (4.75). However, at pH values of less than 4.0, the buffering capacity of acetic acid is substantially less. Yet a further benefit of the invention is that, by conducting the enzymatic hydrolysis and fermentation at a lower pH than in conventional processes, the possibility of microbial contamination is reduced.

Moreover, the invention may result in the production of significantly less inorganic salt than in conventional processes. Since the pH during acid pretreatment is higher than in conventional processes, less acid is present in the pretreated feedstock to form salts with the alkali subsequently added prior to enzymatic hydrolysis. Further, since less alkali is added to the acid pretreated feedstock, less alkali is available to react and form salts with the acid in the pretreated feedstock. Reducing the amount of inorganic salts produced during the process is advantageous as it reduces or eliminates the expense associated with their processing and disposal.

According to one embodiment of the invention, the lignocellulosic feedstock is selected from the group consisting of corn stover, soybean stover, corn cobs, rice straw, rice hulls, corn fiber, wheat straw, barley straw, oat straw, oat hulls and combinations thereof. After combining the feedstock with water, the pH of the resulting feedstock slurry may be between about 6.0 and about 10.0. Preferably, the lignocellulosic feedstock is subjected to size reduction prior to pretreatment so that at least about 90% by weight of the particles produced from the size reduction have a length less than between about 1/16 and about 4 in.

The pretreating may be conducted to hydrolyze at least a portion of hemicellulose present in the feedstock and increase accessibility of cellulose in the feedstock to hydrolysis with cellulase enzymes. Hydrolysis of the hemicellulose produces sugar monomers selected from the group consisting of xylose, glucose, arabinose, mannose, galactose and a combination thereof. The pretreating is preferably conducted at a temperature of between about 160° C. to about 280° C. and the pressure of the pretreatment may be between about 50 psig and 700 psig. Pretreatment may be conducted for between 6 seconds and 3600 seconds. The acid for the pretreatment may be sulfuric acid.

The cellulase enzymes used in the enzymatic hydrolysis preferably comprise cellobiohydrolases (CBHs), endoglucanases (EGs) and β-glucosidase.

The steps of enzymatically hydrolyzing and fermenting may be conducted in the presence of acetic acid originating from the step of pretreating.

Without being limiting, the fermentation product may be an alcohol such as ethanol or butanol. The alcohol may be recovered by distillation. The fermentation product may also be an organic acid, an example of which is lactic acid. Lactic acid may be recovered by liquid-liquid extraction.

It should be understood that the foregoing numerical ranges are approximations. For example, the pH of pretreatment may be 3.6, while enzymatic hydrolysis, fermentation and distillation may each be conducted at a pH of 4.1.

### BRIEF DESCRIPTION OF THE DRAWINGS

These and other features of the invention will become more apparent from the following description in which reference is made to the appended drawings wherein:

FIGS. 1A and 1B are figures showing the minimum and maximum pH of feedstock fed to a lignocellulosic conversion process and the minimum and maximum pH values employed during acid pretreatment, enzymatic hydrolysis, fermentation and distillation in a conventional process to produce ethanol (FIG. 1A) and in an embodiment of the present invention (FIG. 1B).

FIGS. 2A and 2B demonstrate the activity (FIG. 2A) and stability (FIG. 2B) of Trichoderma reesei whole cellulase at reduced pH. The pH profile of the cellulase was measured in a turbidometric assay of insoluble cellulose over a range of pH values. The stability was measured by incubating a volume of the cellulase at pH 3.7, periodically removing a small sample, and assaying its activity at pH 5.0 in the turbidometric assay.

FIGS. 3A and 3B show the glucose yields from the hydrolysis of pretreated feedstock resulting from pretreatment at the pH values indicated. In FIG. 3A, the initial dose of cellulase was 25 mg per gram of cellulose and additional amounts of cellulase (250 mg/g cellulase) were added at the indicated time point during the hydrolysis progression. In FIG. 3B the initial enzyme dosage was increased to 125 mg of cellulase per gram of cellulose.

### DETAILED DESCRIPTION OF THE INVENTION

The following description is of an embodiment by way of example only and without limitation to the combination of features necessary for carrying the invention into effect.

The feedstock for the process is a lignocellulosic material. By the term “lignocellulosic feedstock”, it is meant any type of plant biomass such as, but not limited to, non-woody plant biomass, cultivated crops such as, but not limited to grasses, for example, but not limited to, C4 grasses, such as switch grass, cord grass, rye grass, miscanthus, reed canary grass, or a combination thereof, sugar processing residues, for example, but not limited to, baggase, such as sugar cane bagasse, beet pulp, or a combination thereof, agricultural residues, for example, but not limited to, soybean stover, corn stover, rice straw, sugar cane straw, rice hulls, barley straw, corn cobs, wheat straw, canola straw, oat straw, oat hulls, corn fiber, or a combination thereof, forestry biomass for example, but not limited to, recycled wood pulp fiber, sawdust, hardwood, for example aspen wood, softwood, or a combination thereof. Furthermore, the lignocellulosic feedstock may comprise cellulosic waste material or forestry waste materials such as, but not limited to, newsprint, cardboard and the like. Lignocellulosic feedstock may comprise one species of fiber or, alternatively, lignocellulosic feedstock may comprise a mixture of fibers that originate from different lignocellulosic feedstocks. In addition, the lignocellulosic feedstock may comprise fresh lignocellulosic feedstock, partially dried lignocellulosic feedstock, fully dried lignocellulosic feedstock, or a combination thereof.

Lignocellulosic feedstocks comprise cellulose in an amount greater than about 20%, more preferably greater than about 30%, more preferably greater than about 40% (w/w). For example, the lignocellulosic material may comprise from about 20% to about 50% (w/w) cellulose, or any amount therebetween. Furthermore, the lignocellulosic feedstock comprises lignin in an amount greater than about 10%, more typically in an amount greater than about 15% (w/w). The lignocellulosic feedstock may also comprise small amounts of sucrose, fructose and starch.

The lignocellulosic feedstock is generally first subjected to size reduction by methods including, but not limited to, milling, grinding, agitation, shredding, compression/expansion, or other types of mechanical action. Size reduction by mechanical action can be performed by any type of equipment adapted for the purpose, for example, but not limited to, hammer mills, tub-grinders, roll presses, refiners and hydrapulpers. At least 90% by weight of the particles produced from the size reduction may have a length less than between about 1/16 and about 4 in. The preferable equipment for the particle size reduction is a hammer mill, a refiner or a roll press as disclosed in WO 2006/026863, which is incorporated herein by reference. Subsequent to size reduction, the feedstock is typically slurried in water. This allows the feedstock to be pumped.

The process of the present invention involves subjecting the lignocellulosic feedstock to an acid pretreatment. The acid pretreatment is intended to deliver a sufficient combination of mechanical and chemical action so as to disrupt the fiber structure of the lignocellulosic feedstock and increase the surface area of the feedstock to make it accessible to cellulase enzymes. Preferably, the acid pretreatment is performed so that nearly complete hydrolysis of the hemicellulose and only a small amount of conversion of cellulose to glucose occurs. The cellulose is hydrolyzed to glucose in a subsequent step that uses cellulase enzymes. Typically a dilute acid, at a concentration from about 0.02% (w/w) to about 5% (w/w), or any amount therebetween, (measured as the percentage weight of pure acid in the total weight of dry feedstock plus aqueous solution) is used for the pretreatment.

The acid may be sulfuric acid, sulfurous acid, hydrochloric acid or phosphoric acid. Preferably, the acid is sulfuric acid.

In accordance with the present invention, the pH of the pretreatment is about 2.0 to about 3.5. This includes all values and subvalues therebetween, including 2.0, 2.1, 2.2, 2.3, 2.4, 2.5, 2.6, 2.7, 2.8, 2.9, 3.0, 3.1, 3.2, 3.3, 3.4 or 3.5. In one embodiment of the invention, the pH is between about 2.25 and about 3.5 or between about 2.5 and about 3.5 or any pH range therebetween. In a further embodiment of the invention, the pH of the pretreatment is between about 2.0 and about 2.5.

The acid pretreatment is preferably carried out at a maximum temperature of about 160° C. to about 280° C. However, it should be understood that, in practice, there will be a time delay in the pretreatment process before the feedstock reaches this temperature range. Thus, the above temperatures correspond to those values reached after sufficient application of heat to reach a temperature within this range. The time that the feedstock is held at this temperature may be about 6 seconds to about 3600 seconds, or about 15 seconds to about 750 seconds or about 30 seconds to about 240 seconds.

As set forth previously, the acid pretreatment pH is higher than that which is typically utilized. Other parameters may be adjusted as required to compensate for the milder pH conditions. For example, if the pretreatment pH is increased by 0.5 pH units, the pretreatment time may be doubled. Alternatively, the temperature may be increased by 10° C. for every increase of 0.5 pH units.

The feedstock may be heated with steam during pretreatment. Without being limiting, one method to carry this out is to use low pressure steam to partially heat the feedstock, which is then pumped to a heating train of several stages.

The pretreatment may be carried out under pressure. For example, the pressure during pretreatment may be between about 50 and about 700 psig or between about 75 and about 600 psig, or any pressure range therebetween. That is, the pretreatment may be carried out at 50, 100, 75, 150, 200, 250, 300, 350, 400, 450, 500, 550, 600, 650 or 700 psig, or any amount therebetween.

An alternative to pumping the feedstock directly into a heating train is to leach the salts, proteins, and other impurities out of the feedstock, as set forth in Griffin et al. in WO 02/070753 (incorporated herein by reference). The feedstock may then be pumped into the heating train.

The pretreatment is generally carried out at a solids consistency of 5% to 30% (w/w). The solids consistency is measured by drying at 105° C. overnight, as familiar to those skilled in the art. Those skilled in the art are aware that a solids consistency below this range introduces excess water into the system, while a solids consistency above this range is generally too difficult to pump.

One method of performing acid pretreatment of the feedstock is steam explosion using the process conditions set out in U.S. Pat. No. 4,461,648 (Foody, which is herein incorporated by reference). Another method of pretreating the feedstock slurry involves continuous pretreatment, meaning that the lignocellulosic feedstock is pumped through a reactor continuously. Continuous acid pretreatment is familiar to those skilled in the art; see, for example, U.S. Pat. No. 5,536,325 (Brink); WO 2006/128304 (Foody and Tolan); and U.S. Pat. No. 4,237,226 (Grethlein), which are each incorporated herein by reference. Additional techniques known in the art may be used as required such as the process disclosed in U.S. Pat. No. 4,556,430 (Converse et al.; which is incorporated herein by reference).

The pH of the pretreatment is measured by removing a sample from the pretreatment process after acid addition and measuring the pH of the sample, as is familiar to those of ordinary skill in the art. The pH can change during pretreatment. The pretreatment pH values referred to herein are the final pH values at the conclusion of pretreatment.

The acid pretreatment produces a composition comprising an acid pretreated feedstock. Sugars produced by the hydrolysis of hemicellulose during pretreatment are generally present in the composition and include xylose, glucose, arabinose, mannose, galactose or a combination thereof.

The aqueous phase of the composition comprising the pretreated feedstock may also contain the acid added during the pretreatment. When sulfuric acid is the acid utilized in the pretreatment, the composition comprising the pretreated feedstock additionally contains sulfate and/or bisulfate salts of potassium, sodium, calcium and possibly magnesium. These salts include potassium sulfate, potassium bisulfate, sodium sulfate, sodium bisulfate, calcium sulfate and magnesium sulfate.

The composition comprising acid pretreated feedstock will also comprise acetic acid produced during acid pretreatment. The concentration of acetic acid in this stream may be between 0.1 and 20 g/L.

Additional organic acids may be liberated during pretreatment, including galacturonic acid, formic acid, lactic acid and glucuronic acid. Pretreatment may also produce dissolved lignin and inhibitors such as furfural and hydroxymethyl furfural (HMF). Accordingly, the composition comprising acid pretreated feedstock may also contain these components.

The enzymatic hydrolysis is conducted at a pH below about 4.0. In one embodiment of the invention, the pH is between about 3.0 and about 4.0. This includes all values therebetween, including 3.0, 3.1, 3.2, 3.3, 3.4, 3.5, 3.6, 3.7, 3.8, 3.9 or 4.0. In another embodiment of the invention, the pH is between about 3.5 and about 4.0.

The pH adjustment prior to enzymatic hydrolysis with cellulase enzymes may involve adding sufficient alkali or acid to adjust the pH of the acid pretreated feedstock to less than about 4.0. The stream comprising alkali or acid may be added in-line to the pretreated feedstock or directly to a hydrolysis vessel.

After pH adjustment of the stream comprising pretreated feedstock, enzymatic hydrolysis is conducted. The enzymatic hydrolysis can be carried out with any type of cellulase enzymes suitable for such purpose and effective at the pH and other conditions utilized, regardless of their source. Among the most widely studied, characterized and commercially produced cellulases are those obtained from fungi of the genera Aspergillus, Humicola, Chrysosporium, Melanocarpus and Trichoderma, and from the bacteria of the genera Bacillus and Thermobifida. Cellulase produced by the filamentous fungi Trichoderma longibrachiatum comprises at least two cellobiohydrolase enzymes termed CBHI and CBHII and at least four EG enzymes. As well, EGI, EGII, EGIII, EG V and EGVI cellulases have been isolated from Humicola insolens (see Lynd et al., 2002, Microbiology and Molecular Biology Reviews, 66(3):506-577 for a review of cellulase enzyme systems and Coutinho and Henrissat, 1999, “Carbohydrate-active enzymes: an integrated database approach.” In Recent Advances in Carbohydrate Bioengineering, Gilbert, Davies, Henrissat and Svensson eds., The Royal Society of Chemistry, Cambridge, pp. 3-12, each of which are incorporated herein by reference).

An acid-stable and thermostable EG from Sulfolobus solataricus has been isolated (Huang et al., 2005, Biochem. Journal, 385:581-588, which is incorporated herein by reference) and could be utilized in the practice of the invention.

An appropriate cellulase dosage can be about 1.0 to about 40.0 Filter Paper Units (FPU or IU) per gram of cellulose, or any amount therebetween. The FPU is a standard measurement familiar to those skilled in the art and is defined and measured according to Ghose (Pure and Appl. Chem., 1987, 59:257-268; which is incorporated herein by reference). A preferred cellulase dosage is about 10 to 20 FPU per gram cellulose.

The conversion of cellobiose to glucose is carried out by the enzyme β-glucosidase. By the term “β-glucosidase”, it is meant any enzyme that hydrolyzes the glucose dimer, cellobiose, to glucose. The activity of the β-glucosidase enzyme is defined by its activity by the Enzyme Commission as EC#3.2.1.21. The β-glucosidase enzyme may come from various sources; however, in all cases, the β-glucosidase enzyme can hydrolyze cellobiose to glucose. The β-glucosidase enzyme may be a Family 1 or Family 3 glycoside hydrolase, although other family members may be used in the practice of this invention. The preferred 3-glucosidase enzyme for use in this invention is the Bgl1 protein from Trichoderma reesei. It is also contemplated that the β-glucosidase enzyme may be modified to include a cellulose binding domain, thereby allowing this enzyme to bind to cellulose.

An example of a β-glucosidase enzyme that can be employed in the practice of the invention is an acid-tolerant β-glucosidase described in co-owned PCT/CA2009/111203, the contents of which are incorporated herein by reference.

The cellulase enzymes and β-glucosidase enzymes may be handled in an aqueous solution or as a powder or granulate. The enzymes may be added to the pretreated feedstock at any point prior to its introduction into a hydrolysis reactor. Alternatively, the enzymes may be added directly to the hydrolysis reactor, although addition of enzymes prior to their introduction into the hydrolysis reactor is preferred for optimal mixing. The enzymes may be mixed into the pretreated feedstock using mixing equipment that is familiar to those of skill in the art.

In practice, the hydrolysis is carried out in a hydrolysis system, which includes multiple hydrolysis reactors. The number of hydrolysis reactors in the system depends on the cost of the reactors, the volume of the aqueous slurry, and other factors. For a commercial-scale ethanol plant, the typical number of hydrolysis reactors may be for example, 4 to 12. In order to maintain the desired hydrolysis temperature, the hydrolysis reactors may be jacketed with steam, hot water, or other heat sources. Preferably, the cellulase hydrolysis is a continuous process, with continuous feeding of pretreated lignocellulosic feedstock and withdrawal of the hydrolyzate slurry. However, it should be understood that batch processes are also included within the scope of the present invention.

Other design parameters of the hydrolysis system may be adjusted as required. For example, the volume of a hydrolysis reactor in a cellulase hydrolysis system can range from about 100,000 L to about 3,000,000 L, or any volume therebetween, for example, between 200,000 and 750,000 L, or any amount therebetween, although reactors of small volume may be preferred to reduce cost. The total residence time of the slurry in a hydrolysis system may be between about 12 hours to about 200 hours, or any amount therebetween, for example, 25 to 100 hours, or 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98 100, 120, 140, 160, 180, 200 hours, or any amount therebetween. The hydrolysis reactors may be unmixed or subjected to light agitation, typically with a maximum power input of up to 0.8 hp/1000 gallons, or may receive heavy agitation of up to 20 hp/1000 gallons.

Following enzymatic hydrolysis of the pretreated feedstock, any insoluble solids present in the resulting sugar stream, including lignin, may be removed using conventional solid-liquid separation techniques prior to any further processing. However, it may be desirable in some circumstances to carry forward both the solids and liquids in the sugar stream for further processing.

The hydrolysis may be a continuous process, with continuous feeding of pretreated feedstock and withdrawal of hydrolysis product. Alternatively, the process is a batch process.

In accordance with the invention, the fermentation is conducted at a pH below about 4.0. For example, the pH of the fermentation may be between about 3.0 and about 4.0. This includes all subranges and values therebetween, including pH values of 3.0, 3.1, 3.2, 3.3, 3.4, 3.5, 3.6, 3.7, 3.8, 3.9 and 4.0. For example, the pH may be between about 3.5 and about 4.0 or between about 3.8 and about 4.0. In one embodiment of the invention, the pH of the fermentation is about the same or less than the pH employed in the enzymatic hydrolysis.

Fermentation of glucose resulting from cellulose hydrolysis may produce one or more of the fermentation products selected from an alcohol, a sugar alcohol, an organic acid and a combination thereof.

In one embodiment of the invention, the fermentation product is an alcohol, such as ethanol or butanol. For ethanol production, fermentation is typically carried out with a Saccharomyces spp. yeast. Glucose and any other hexoses present in the sugar stream may be fermented to ethanol by wild-type Saccharomyces cerevisiae, although genetically modified yeasts may be employed as well, as discussed below. The ethanol may then be distilled to obtain a concentrated ethanol solution. Butanol may be produced from glucose by a microorganism such as Clostridium acetobutylicum and then concentrated by distillation.

In addition to the glucose resulting from enzymatic hydrolysis, sugars liberated during pretreatment, namely xylose, arabinose, mannose, galactose, or a combination thereof, will typically also be present in the stream sent to fermentation.

Xylose and arabinose may also be fermented to ethanol by a yeast strain that naturally contains, or has been engineered to contain, the ability to ferment these sugars to ethanol. Examples of microbes that have been genetically modified to ferment xylose include recombinant Saccharomyces strains into which has been inserted either (a) the xylose reductase (XR) and xylitol dehydrogenase (XDH) genes from Pichia stipitis (U.S. Pat. Nos. 5,789,210, 5,866,382, 6,582,944 and 7,527,927 and European Patent No. 450530) or (b) fungal or bacterial xylose isomerase (XI) gene (U.S. Pat. Nos. 6,475,768 and 7,622,284). Examples of yeasts that have been genetically modified to ferment L-arabinose include, but are not limited to, recombinant Saccharomyces strains into which genes from either fungal (U.S. Pat. No. 7,527,951) or bacterial (WO 2008/041840) arabinose metabolic pathways have been inserted.

Organic acids that may be produced during the fermentation include lactic acid, citric acid, ascorbic acid, malic acid, succinic acid, pyruvic acid, hydroxypropanoic acid, itaconoic acid and acetic acid. In a non-limiting example, lactic acid is the fermentation product of interest. The most well-known industrial microorganisms for lactic acid production from glucose are species of the genera Lactobacillus, Bacillus and Rhizopus.

In one embodiment of the invention, the microorganism utilized in the fermentation is acid-tolerant. Any known or developed microorganism can be employed in the practice of the invention. For example, an acetic acid tolerant galactose-fermenting Saccharomyces cerevisiae yeast strain has been isolated from spent sulfite liquor (a byproduct of sulfite pulping) by adaptation techniques, as set forth in Lindén et al. (Applied and Environmental Microbiology, 1992, 58(5):1661-1669). Moreover fungi from the genus Rhizopus or yeast transformed with lactic dehydrogenase such as Kluyveromyces, Saccharomyces, Torulaspora and Zygosaccharomyces (WO 99/14335) have been known to effect the conversion of glucose to lactic acid under acidic conditions. US 2006/0094093 discloses acid-tolerant homolactic bacteria, including Lactobacillus strains.

Moreover, xylose and other pentose sugars may be fermented to xylitol by yeast strains selected from the group consisting of Candida, Pichia, Pachysolen, Hansenula, Debaryomyces, Kluyveromyces and Saccharomyces. Bacteria are also known to produce xylitol, including Corynebacterium sp., Enterobacter liquefaciens and Mycobacterium smegmatis.

In practice, the fermentation is typically performed at or near the temperature and pH optimum of the fermentation microorganism. A typical temperature range for the fermentation of glucose to ethanol using Saccharomyces cerevisiae is between about 25° C. and about 35° C., although the temperature may be higher if the yeast is naturally or genetically modified to be thermostable. The dose of the fermentation microorganism will depend on other factors, such as the activity of the fermentation microorganism, the desired fermentation time, the volume of the reactor and other parameters. It should be appreciated that these parameters may be adjusted as desired by one of skill in the art to achieve optimal fermentation conditions.

The fermentation may also be supplemented with additional nutrients required for the growth of the fermentation microorganism. For example, yeast extract, specific amino acids, phosphate, nitrogen sources, salts, trace elements and vitamins may be added to the hydrolyzate slurry to support their growth.

The fermentation may be conducted in batch, continuous or fed-batch modes with or without agitation. Preferably, the fermentation reactors are agitated lightly with mechanical agitation. A typical, commercial-scale fermentation may be conducted using multiple reactors. The fermentation microorganisms may be recycled back to the fermentor or may be sent to distillation without recycle.

By the term “recovering”, it is meant that the fermentation product is obtained in a more purified and/or concentrated form than that in the fermentation broth. The recovery may be carried out by any suitable technique known to those of ordinary skill in the art, and includes distillation for fermentation products that have a higher or lower boiling point than water, such as ethanol and butanol, or techniques such as liquid-liquid extraction for lactic acid.

If ethanol or butanol is the fermentation product, the recovery is carried out by distillation, typically with further concentration by molecular sieves or membrane extraction.

The fermentation broth that is sent to distillation is a dilute alcohol solution containing solids, including unconverted cellulose, and any components added during the fermentation to support growth of the microorganisms.

The pH of the fermentation broth sent to distillation is less than 4.0. For example, the pH may be between about 3.0 and about 4.0, including all values and subranges therebetween. That is, the pH of the fermentation broth may be 3.0, 3.1, 3.2, 3.3, 3.4, 3.5, 3.6, 3.7, 3.8, 3.9 or 4.0. In one embodiment, the pH is between about 3.5 and about 4.0 or between about 3.8 and about 4.0.

In one example of the invention, the pH of the fermentation broth sent to distillation is about the same or less than the pH employed in the enzymatic hydrolysis.

Microorganisms are potentially present during the distillation depending upon whether or not they are recycled during the fermentation. The broth is preferably degassed to remove carbon dioxide and then pumped through one or more distillation columns to separate the alcohol from the other components in the broth. The mode of operation of the distillation system depends on whether the alcohol has a lower or a higher boiling point than water. Most often, the alcohol has a lower boiling point than water, as is the case when ethanol is distilled.

In those embodiments where ethanol is concentrated, the column(s) in the distillation unit is preferably operated in a continuous mode, although it should be understood that batch processes are also encompassed by the present invention. Heat for the distillation process may be introduced at one or more points either by direct steam injection or indirectly via heat exchangers. The distillation unit may contain one or more separate beer and rectifying columns, in which case dilute beer is sent to the beer column where it is partially concentrated. From the beer column, the vapour goes to a rectification column for further purification. Alternatively, a distillation column is employed that comprises an integral enriching or rectification section.

After distillation, the water remaining may be removed from the vapour by a molecular sieve resin, by membrane extraction, or other methods known to those of skill in the art for concentration of ethanol beyond the 95% that is typically achieved after distillation. The vapour may then be condensed and denatured.

An aqueous stream(s) remaining after ethanol distillation and containing solids, referred to herein as “still bottoms”, is withdrawn from the bottom of one or more of the column(s) of the distillation unit. This stream will contain inorganic salts, unfermented sugars and organic salts.

When the alcohol has a higher boiling point than water, such as butanol, the distillation is run to remove the water and other volatile compounds from the alcohol. The water vapor exits the top of the distillation column and is known as the “overhead stream”.

Product recovery and purification of organic acids, such as lactic acid, often requires that the acid is in its undissociated form. This is the dominant species at pH values below the pKa of the acid (pKa of lactic acid is 3.86). Lactic acid may be recovered by any one of a number of known methods, including extraction from solution. Extraction can be carried out using a tertiary amine-containing extractant. An example of a suitable extractant is a solution of Alamine® 336 in octyl alcohol. Other methods that may be used to isolate the lactic acid include contacting the solution with a solid adsorbent, such as an ion exchange resin, distilling off a lactic acid containing fraction, or removal via membrane separation. (See US 2006/0094093 and US 2004/0210088, which are incorporated herein by reference). The solution enriched in lactic acid may be further processed to separate out lactate salt such as by extraction, crystallization, membrane separation or anion exchange.

### EXAMPLES

### Example 1

### Comparative Example Showing the Acid and Alkali Demand of Conventional Processes to Produce Ethanol Verses a Process of the Present Invention

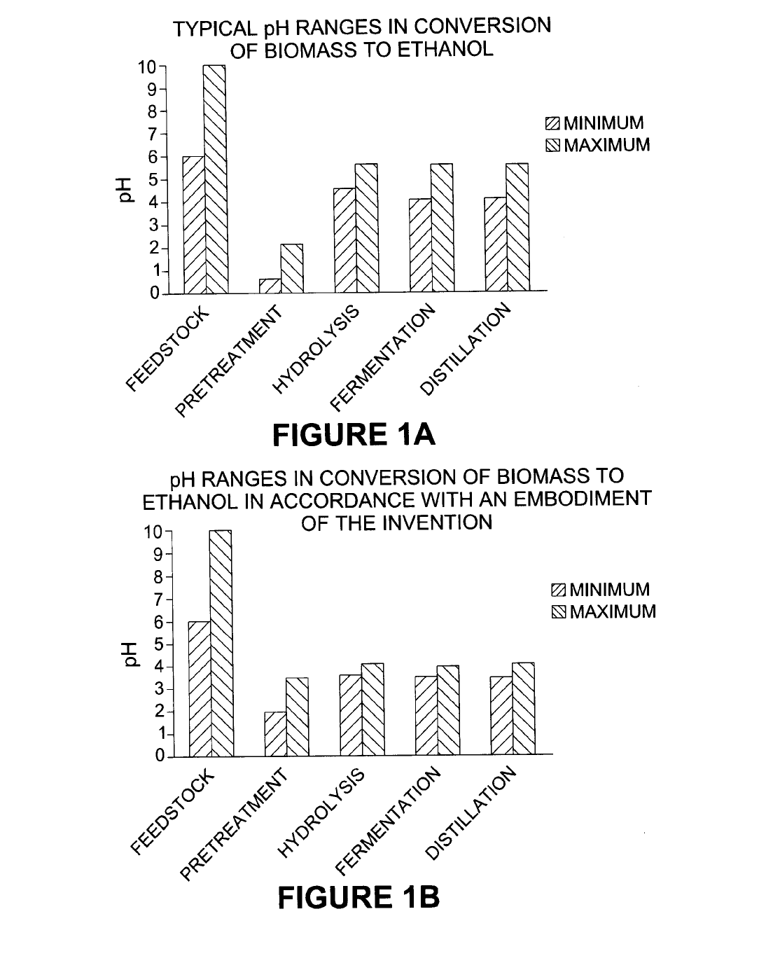

FIG. 1A shows the minimum and maximum pH of lignocellulosic feedstocks used to produce ethanol as well as the pH values that are conventionally employed in each stage of a process employing acid pretreatment, enzymatic hydrolysis, fermentation and distillation.

As shown in the conventional process of FIG. 1A, the pH of the incoming lignocellulosic feedstock is between about 6.0 and 8.0. The pH of the feedstock then is decreased with acid, such as sulfuric acid, to a pH between about 0.5 and 2.0. Acid pretreatment is then conducted at a temperature and for a time sufficient to hydrolyze the hemicellulose component of the feedstock with limited hydrolysis of cellulose. Alkali is subsequently added to the acidic, pretreated feedstock to achieve a pH in the range of 4.5 to 5.5 for cellulase enzymes. Enzymatic hydrolysis of the pretreated feedstock produces a glucose stream. The pH of the glucose stream may then be adjusted to a value that is amenable to most ethanol fermentations and this is usually between 4.0 and 5.5 for the yeast that are commonly used in this stage, such as Saccharomyces cerevisiae. The ethanol in the fermentation broth produced from the fermentation is distilled to produce a concentrated ethanol solution. The fermentation broth or “beer” fed to the distillation is generally at a pH between 4.0 and 5.5 and thus may not require a pH adjustment.

For the conventional process depicted in FIG. 1A, the amount of chemical required for each stage is provided in Table 1. For the calculations, it was assumed that the feedstock was wheat straw at 12% consistency in pretreatment and hydrolysis and equivalent solids basis in fermentation. The straw consists of 5.0% acetyl that is liberated as acetic acid in the pretreatment.

TABLE 1
Chemical consumption for conventional processes for producing
ethanol from lignocellulosic feedstocks by acid pretreatment,
enzymatic hydrolysis, and fermentation.

| Process stage | | | | | |
|---|---|---|---|---|---|
| | | | | Acid for | |
| | | Sulfuric acid or | | neutralization | |
| Prior | Stage | sodium hydroxide | | of feedstock | Total |
| pH | pH | Mol/L | Kg/t | Kg/t | Kg/t |

Pretreatment (sulfuric acid usage)

| | | | | | | |
|---|---|---|---|---|---|---|
| Minimum | 7.5 | 2.0 | 0.008 | 6.5 | 8.0 | 14.5 |
| acid | | | | | | |
| Maximum | 7.5 | 0.5 | 0.4 | 326.7 | 24.0 | 350.7 |
| acid | | | | | | |

Hydrolysis/fermentation/distillation (sodium hydroxide usage)

| | | | | | | |
|---|---|---|---|---|---|---|
| Minimum | 2.0 | 4.5 | 0.04 | 13.3 | Not | 13.3 |
| base | | | | | applicable | |
| Maximum | 0.5 | 5.5 | 0.89 | 296.7 | Not | 296.7 |
| base | | | | | applicable | |

Total for process

| | |
|---|---|
| Minimum | 14.5 kg/t sulfuric acid |
| | 13.3 kg/t sodium hydroxide |
| Maximum | 350.7 kg/t sulfuric acid |
| | 296.7 kg/t sodium hydroxide |

TABLE 2
Chemical consumption for producing ethanol from lignocellulosic
feedstocks by acid pretreatment, enzymatic hydrolysis
and fermentation at pH ranges of the invention.

| Process stage | | | | | |
|---|---|---|---|---|---|
| | | | | Acid for | |
| | | Sulfuric acid or | | neutralization | |
| Prior | Stage | sodium hydroxide | | of feedstock | Total |
| pH | pH | Mol/L | Kg/t | Kg/t | Kg/t |

Pretreatment (sulfuric acid usage)

| | | | | | | |
|---|---|---|---|---|---|---|
| Minimum | 7.5 | 3.5 | 0.0002 | 0.16 | 5.0 | 5.16 |
| acid | | | | | | |
| Maximum | 7.5 | 2.0 | 0.008 | 6.5 | 8.0 | 14.5 |
| acid | | | | | | |

Hydrolysis/fermentation/distillation (sodium hydroxide usage)

| | | | | | | |
|---|---|---|---|---|---|---|
| Minimum | 3.5 | 3.5 | 0.0 | 0.0 | Not | 0.0 |
| base | | | | | applicable | |
| Maximum | 2.0 | 4.0 | 0.034 | 11.3 | Not | 11.3 |
| base | | | | | applicable | |

Total for process

| | |
|---|---|
| Minimum | 5.16 kg/t sulfuric acid |
| | 0.0 kg/t sodium hydroxide |
| Maximum | 14.5 kg/t sulfuric acid |
| | 11.3 kg/t sodium hydroxide |

FIG. 1B shows the pH ranges of a process according to an embodiment of the present invention. The pH of the incoming feedstock is between about 6.0 and 8.0 and then is decreased with acid, such as sulfuric acid, to a pH between about 2.0 and 3.5. Acid pretreatment is then conducted at a temperature and for a time sufficient to hydrolyze the hemicellulose component of the feedstock with limited hydrolysis of cellulose. Alkali is then added to the acidic, pretreated feedstock to achieve a pH within the range of 3.5 and 4.0. Enzymatic hydrolysis of the pretreated feedstock produces a glucose stream. The pH of the glucose stream sent to fermentation to produce ethanol with Saccharomyces cerevisiae is between 3.5 and 4.0. The ethanol in the fermentation broth produced from the fermentation is subsequently distilled to produce a concentrated ethanol solution. The fermentation broth fed to the step of distillation is also at a pH between 3.5 and 4.0.

In the process depicted in FIG. 1B, the amount of chemical required for each stage is provided in Table 2. Similar to the calculations for Table 1, it was assumed that the feedstock was wheat straw at 12% consistency in pretreatment and hydrolysis and equivalent solids basis in fermentation. The straw consists of 5.0% acetyl that is liberated as acetic acid in the pretreatment.

Comparing the chemical demand in Table 1 of a conventional process to the chemical demand in Table 2 calculated based on the pH ranges of embodiments of the invention, it can be seen that the chemical demand of the latter is significantly less than that of the conventional process. Notably, even the total minimum levels of sulfuric acid and sodium hydroxide usage in Table 1 are higher than the respective total maximum levels in Table 2.

### Example 2

### The Activity and Stability of Trichoderma Cellulase at Reduced pH

Wheat straw was pretreated using dilute acid steam explosion (U.S. Pat. No. 4,461,648, which is incorporated herein by reference) and delignified using hypochlorite bleaching and caustic extraction. The delignified material was slurried in water to a final concentration of 1.8 g cellulose/L and homogenized with a rotor-stator homogenizer. It was then degassed under vacuum for 5 minutes with constant stirring prior to use in the assay.

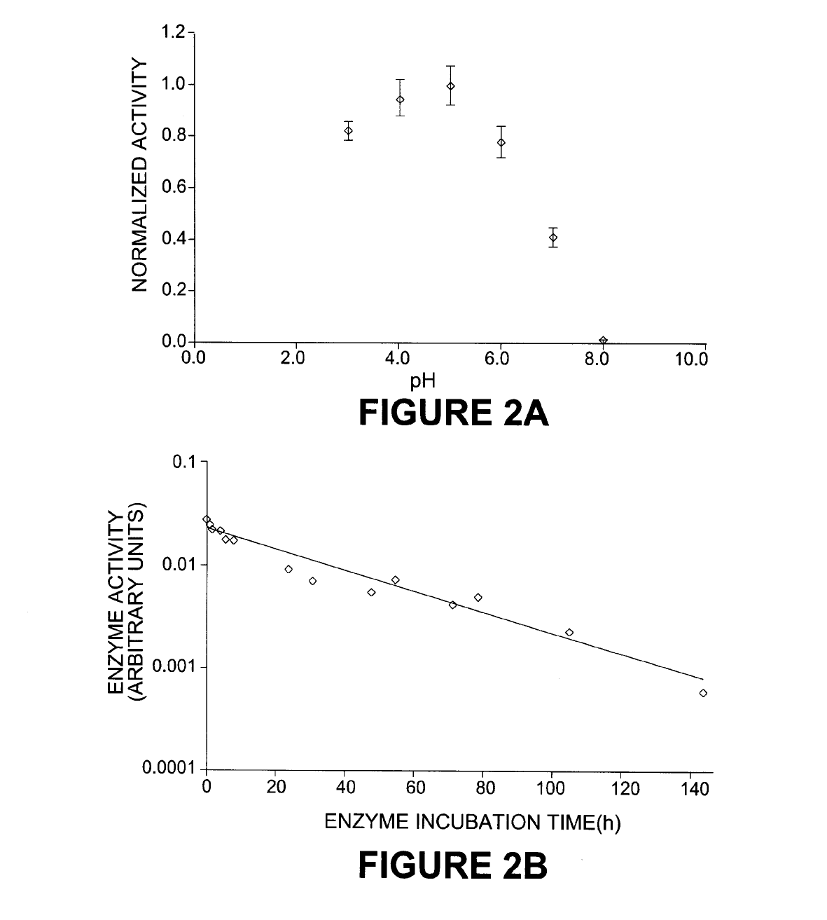

The slurry was further diluted to 0.6 g/L cellulose using concentrated citrate-phosphate buffer having a working buffer concentration of 50 mM. Samples were prepared in methacrylate cuvettes to a final volume of 3 mL. Samples were prepared over the pH range of 3.0 to 8.0 in increments of 1 pH unit. The absorbance of each slurry at 600 nm and 50° C. was monitored in a Cary300 spectrophotometer (Varian) with a temperature-controlled heating block. Samples were first incubated and monitored for 5 minutes to verify a stable background of apparent absorbance. Trichoderma reesei whole cellulase was then added at a dose of 50 mg of enzyme per g of cellulose and the apparent absorbance as a function of time was monitored. The action of the enzyme on the insoluble cellulose results in a decrease in apparent absorbance and the slope of this decrease, calculated over 2-5 minutes after enzyme addition, is proportionate to the enzyme activity. Triplicate data sets were collected for all samples and the activity of the enzyme as a function of pH was plotted, normalizing the results to the highest activity observed (FIG. 2A).

For the stability assay, Trichoderma reesei whole cellulase was diluted to 1 mg/mL in 50 mM citrate-phosphate buffer, pH 3.7, which had been pre-warmed to 50° C. The sample, of total volume 50 mL, was incubated at this temperature with 250 rpm orbital shaking for a total of 96 h. Samples of 1.5 mL were removed 0, 0.5, 1, 2, 4, 6, 8, 24, 32, 48, 56, 72, 78, and 96 h after the addition of the enzyme to the buffer. The cellulase activity of each time point was measured using the turbidometric assay as described above, with the differences that a sufficient quantity of the time point solution was added to the cuvette to achieve a dose of 180 mg of enzyme per gram of cellulose and that the activity assay was carried out at pH 5.0. The inactivation of the enzyme, evidenced as a decrease in activity over time, was modeled using a first order exponential decay (FIG. 2B).

Trichoderma whole cellulase maintains >80% of its maximum activity between pH 3.0 and 4.0 and has a mean lifetime (the inverse of its inactivation rate) of 43 h at pH 3.7. Collectively, these data demonstrate that extended hydrolysis at pH values less than 4.0 are feasible. The enzyme dose can be selected to give the requisite conversion within the active lifetime of the enzyme, or multiple doses of enzyme can be added if longer hydrolyses are desired.

### Example 3

### Enzymatic Hydrolysis of Pretreated Feedstock with Trichoderma Cellulase at Reduced pH

Wheat straw was pretreated using dilute acid steam explosion (U.S. Pat. No. 4,461,648, which is incorporated herein by reference). The resulting pretreated feedstock solids contained 46.2% cellulose and the slurry of pretreated wheat straw contained 7.35% undissolved solids (% UDS). For each assay, 70 grams of slurry was adjusted to the target pH with a 15 wt % NaOH solution. Cellulase was added to the slurry at a dosage of 25 mg of cellulase per gram of cellulose (mg/g) and the mixture incubated at 50° C. with orbital shaking at 250 rpm for 120 hours. Samples (500 μL) were removed at selected time points, boiled for 10 minutes to deactivate the cellulase, and then stored at 4° C. for later analysis. After 120 hours, an additional 250 mg cellulase per gram of cellulose was added to the assay flasks and the hydrolysis was continued for a total of 168 hrs.

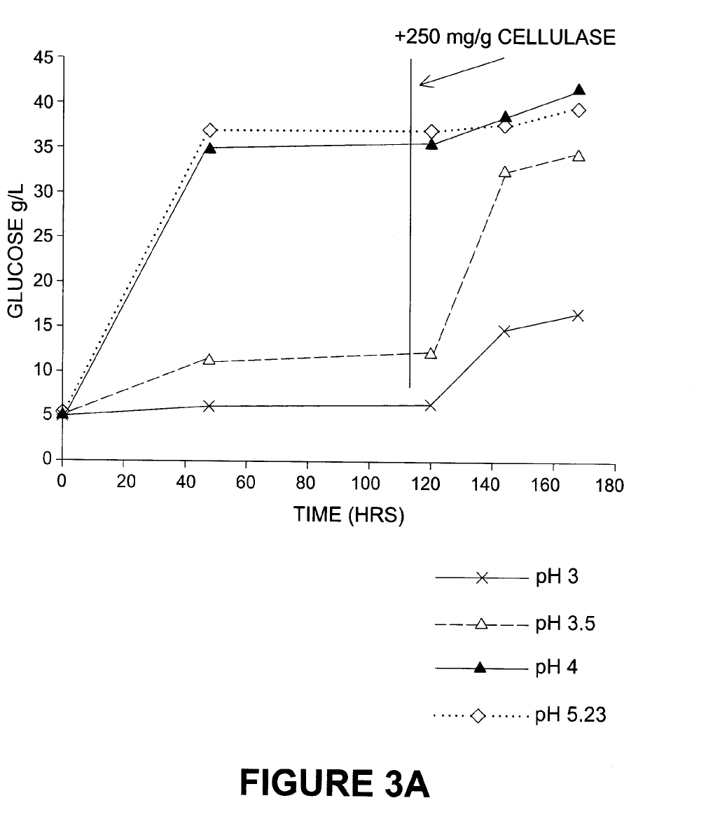

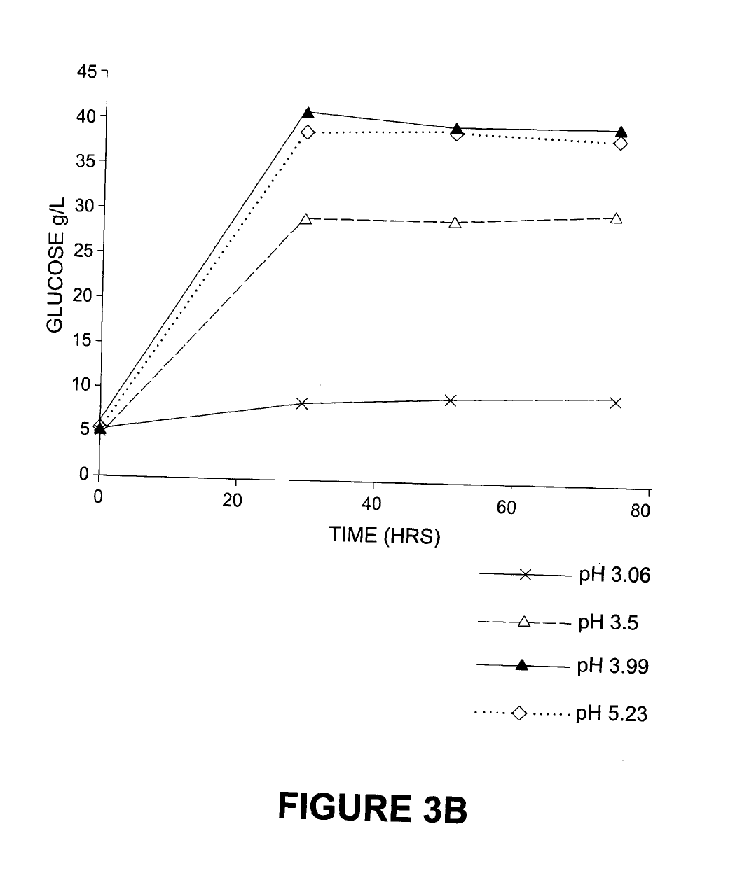

In a second series of assays, cellulase was added to the pretreated feedstock slurry at a dosage of 125 mg of cellulase per gram of cellulose using the procedures above but for a total reaction time of 74 hrs. For all assays, the cellulase activity was measured by determining the glucose produced at selected time points and plotted as a function of time (FIG. 3). Glucose was measured using a Dionex HPLC and a PA1 column. The results shows that the glucose yields from the hydrolysis of pretreated feedstock at reduced pH can be improved by introducing additional amounts of cellulase during the hydrolysis (FIG. 3A) or by increasing the initial enzyme dosage (FIG. 3B).

### Example 4

### Fermentation of Sugars Produced by Trichoderma Cellulase from Hydrolyses of Pretreated Feedstocks at Reduced pH

The sugars from Example 3 obtained from the hydrolysis at 125 mg cellulase per gram of cellulose dosages were fermented using commercially available Superstart™ yeast at 25 g/L (Lallemand Ethanol Technology). For the fermentations, 50 mL were incubated in shakeflasks at 30° C. with 250 rpm orbital shaking. The initial pH of the fermentation and the amount of ethanol produced after 3.5 hours in each flask is tabulated in Table 3. The data shows that yeast fermentation of sugars produced at reduced hydrolysis pH are easily converted to ethanol.

TABLE 3
Fermentation results

| Hydrolysis | Fermentation | time | Glucose | Ethanol | % of |
|---|---|---|---|---|---|
| pH | pH | (hrs) | (g/L) | (g/L) | theoretical |
| 3.06 | 3.16 | 0 | 11.4 | 0.2 | |
| | | 3.5 | 0.0 | 2.7 | 46.5 |
| 3.5 | 3.54 | 0 | 32.6 | 0.2 | |
| | | 3.5 | 0.0 | 13.2 | 79.3 |
| 3.99 | 4.04 | 0 | 44.3 | 0.3 | |
| | | 3.5 | 0.6 | 18.7 | 83 |

### Example 5

### Pretreatment of Lignocellulosic Feedstocks at pH Ranges of the Invention

Acidified straw was prepared by combining 20 g of ½ inch straw (moisture content of 4.5%) with 387 g of deionized water and adding 10 wt % H₂SO₄ until the target pH was reached. Before acid addition, the pH measured was 9.3. After each acid addition, about 5-10 minutes and was needed for the acid to react with the alkali leached from the feedstock and for a stable pH to be reached. Once at the target pH, the acidified straw slurry was transferred to a pre-warmed (105° C.) autoclave. The target autoclave temperature of 136-138° C. was held for 90 minutes. The results in Table 4 demonstrate that compared to a conventional pretreatment at pH 1.25, pretreatment at the pH ranges of the invention can reduce the acid consumption by about 89% or greater.

TABLE 4
Relative acid requirements at different pretreatment pH values

| | | | Relative amount |
|---|---|---|---|
| pretreatment pH | H₂SO_{4,} g | g H₂SO₄/g straw | of acid used |
| 1.25 | 4.65 | 0.47 | 100 |
| 2.05 | 0.53 | 0.053 | 11.33 |
| 2.85 | 0.30 | 0.030 | 6.37 |
| 3.40 | 0.25 | 0.025 | 5.33 |

### Example 6

### Pretreatment of Lignocellulosic Feedstocks at pH Ranges of the Invention

A hundred grams of acidified straw was prepared by combining 10 g of blended straw (moisture content of 6.1%) with 90 g of deionized water and adding 10 wt % H₂SO₄ until the target pH was reached. Straw blending took place in a kitchen blender for 1-2 minutes. Before acid addition, the pH measured was 8.7. After each acid addition, about 5-10 minutes was needed for the acid to react with the alkali leached from the feedstock and for a stable pH to be reached. Once at the target pH, the acidified straw was transferred to a pre-warmed (105° C.) autoclave. The target autoclave temperature of 136-138° C. was held for 81 minutes. For each target pH (1.25, 2 and 3.5), duplicate flasks were prepared. Compared to the conventional pretreatment pH, the pretreatment carried out at the higher pH of the invention required about 86-87% less sulfuric acid.

TABLE 5
Relative acid requirements at different pretreatment pH values

| | | | Relative amount of |
|---|---|---|---|
| Acidified straw pH | H₂SO_{4,} g | g H₂SO₄/g straw | acid used* |
| 1.26 | 0.67 | 0.067 | 100 |
| 1.26 | 0.77 | 0.077 | 100 |
| 2.05 | 0.19 | 0.019 | 13.36 |
| 2.03 | 0.20 | 0.02 | 13.56 |
| 3.09 | 0.12 | 0.012 | 8.51 |
| 3.10 | 0.13 | 0.013 | 9.07 |
| 3.46 | 0.10 | 0.01 | 6.85 |
| 3.47 | 0.10 | 0.01 | 6.78 |

*% relative to average of H₂SO₄ used at pH 1.26

After pretreatment, the contents of the sample were adjusted to 100 grams by adding deionized water to compensate for the small amount of moisture lost during the autoclave step. One set of flasks were used to determine the % UDS of the pretreated slurry. NaOH was added to the second set of flasks to reach the pH targeted for the hydrolysis of the invention. The amount of NaOH required to reach the targeted pH is given in Table 6. The alkali needed for the process of the invention was reduced by about 92-100% compared to that for the conventional process.

TABLE 6
Alkali requirements for pH adjustment prior to
enzymatic hydrolysis

| Acidified | % UDS of | Target hydrolysis | g of NaOH/ |
|---|---|---|---|
| straw pH | pretreated straw | pH | g straw |
| 1.26 | 5.62 | | |
| 1.26 | | 5.08 | 0.096 |
| 2.05 | 6.64 | | |
| 2.03 | | 3.07 | 0.008 |
| 3.09 | 7.67 | | |
| 3.10 | | 3.99 | 0.002 |
| 3.46 | 8.47 | | |
| 3.47 | | 4.39 | 0.00 |

1. A method for obtaining a fermentation product from a lignocellulosic feedstock comprising:
(i) pretreating the lignocellulosic feedstock with acid at a pH between about 2.0 and about 3.5 to produce a composition comprising an acid pretreated feedstock;
(ii) enzymatically hydrolyzing the acid pretreated feedstock with cellulases and β-glucosidase to produce glucose;
(iii) fermenting the glucose so produced with microorganisms to produce a fermentation broth comprising the fermentation product; and
(iv) recovering the fermentation product from the fermentation broth,
wherein the pH during each of the steps of enzymatically hydrolyzing, fermenting and recovering is between about pH 3.0 and about 4.0 and wherein the pH during fermenting is greater than or equal to the pH during enzymatically hydrolyzing and the pH during recovering is greater than or equal to the pH during the fermenting.

2. The method according to claim 1, wherein the steps of fermenting and recovering are each conducted at a pH between about 3.5 and about 4.0.

3. The method according to claim 1, wherein the acid used for pretreating said lignocellulosic feedstock is sulfuric acid.

4. The method according to claim 1, wherein said fermentation product is an alcohol.

5. The method according to claim 4, wherein said recovering comprises distillation.

6. The method according to claim 4, wherein said alcohol is ethanol.

7. The method according to claim 4, wherein the alcohol is butanol.

8. The method according to claim 1, wherein the fermentation product is an organic acid.

9. The method according to claim 8, wherein the organic acid is lactic acid.

10. The method according to claim 8, wherein the recovering comprises liquid-liquid extraction.

11. The method according to claim 1, wherein the pretreating comprises hydrolyzing at least a portion of hemicellulose present in said feedstock and increase accessibility of cellulose in said feedstock to being hydrolyzed with said cellulase enzymes.

12. The method according to claim 11, wherein the hydrolyzing produces sugar monomers selected from the group consisting of xylose, glucose, arabinose, mannose, galactose and a combination thereof.

13. The method according to claim 1, wherein the pretreating is conducted at a temperature of between about 160° C. to about 280° C.

14-15. (canceled)

16. The method according to claim 1, wherein the cellulase enzymes comprise cellobiohydrolases (CBHs) and endoglucanases (EGs).

17. The method according to claim 1, wherein the lignocellulosic feedstock is selected from the group consisting of corn stover, soybean stover, corn cobs, rice straw, rice hulls, corn fiber, wheat straw, barley straw, canola straw, oat straw, oat hulls and combinations thereof.

18. The method according to claim 1, wherein the pH of the lignocellulosic feedstock, after slurrying, is between about 6.0 and about 8.0.

19. The method according to claim 1, wherein the lignocellulosic feedstock is subjected to size reduction prior to pretreatment and wherein at least about 90% by weight of the particles produced from the size reduction have a length less than between about 1/16 and about 4 in.

20. The method according to claim 1, wherein the steps of enzymatically hydrolyzing and fermenting are conducted in the presence of acetic acid originating from the step of pretreating.

21. A method for obtaining a fermentation product from a lignocellulosic feedstock comprising:
(i) pretreating the lignocellulosic feedstock with acid at a pH between about 2.5 and about 3.5 to produce a composition comprising an acid pretreated feedstock;
(ii) enzymatically hydrolyzing the acid pretreated feedstock with cellulases and β-glucosidase to produce glucose;
(iii) fermenting the glucose so produced with microorganisms to produce a fermentation broth comprising the fermentation product; and
(iv) recovering the fermentation product from the fermentation broth,
wherein the pH during each of the steps of enzymatically hydrolyzing, fermenting and recovering is between about pH 3.0 and about 4.0.

22. A method for obtaining a fermentation product from a lignocellulosic feedstock comprising:
(i) pretreating the lignocellulosic feedstock with acid at a pH between about 2.0 and about 3.5 to produce a composition comprising an acid pretreated feedstock;
(ii) enzymatically hydrolyzing the acid pretreated feedstock with cellulases and β-glucosidase to produce glucose; and
(iii) fermenting the glucose so produced with microorganisms to produce a fermentation broth comprising the fermentation product;
wherein the steps of enzymatically hydrolyzing and fermenting are each conducted at a pH of between about 3.0 about 4.0.

23. (canceled)
